(19) United States
(12) Patent Application Publication
(10) Pub. No.: US 2026/0244718 A1
(43) Pub. Date: Aug. 20, 2026

(54) DETERMINISTIC FINGERPRINTING OF AI AGENTS

(71) Applicant: Drive Health AI, Gilbert, AZ (US)

(72) Inventors: Alexander SICULAR, Dallas, TX (US); Kevin LONGORIA, Gilbert, AZ (US); Rajkumar KHANDELWAL, Chandler, AZ (US)

(73) Assignee: Drive Health AI, Gilbert, AZ (US)

(21) Appl. No.: 19/395,665

(22) Filed: Nov. 20, 2025

Related U.S. Application Data
(60) Provisional application No. 63/760,607, filed on Feb. 19, 2025.

(51) Int. Cl. G06F 21/16; G06F 8/60; G06F 21/60

(52) U.S. Cl. CPC G06F 21/16; G06F 8/60; G06F 21/602

(57) ABSTRACT

Systems and methods for fingerprinting artificial intelligence (AI) agents are disclosed. A system can identify an AI agent that includes a plurality of components, the AI agent being associated with an identifier for a manufacturer of the AI agent. The system can encrypt the plurality of components to obtain a plurality of component identifiers for the AI agent. The system can assemble the plurality of component identifiers and the identifier for the manufacturer into a structure of a metadata file. The system can encrypt the metadata file to generate a fingerprint of the AI agent. The system can provide the fingerprint and metadata file for validation of an instance of the AI agent by a user.

### CROSS-REFERENCE TO RELATED APPLICATIONS

The present application claims the benefit of and priority to U.S. Provisional Application No. 63/670,607, filed Feb. 19, 2025, the disclosure of which is incorporated herein by reference in its entirety.

### BACKGROUND

Artificial intelligence (AI) systems, including agentic AI systems, can include numerous interdependent software and data components that interact to perform computational tasks. Establishing consistency, authenticity, and integrity across these components can be difficult, particularly when systems are modified, updated, or deployed in distributed environments. For example, AI systems may be expected to achieve target performance criteria, but it can be computationally demanding to reliably determine that an AI system meets such criteria; the performance of the AI system can be non-deterministic, further limiting the ability to ensure that a given deployment of the AI system will meet the criteria.

### SUMMARY

AI systems, including agentic systems, can be deployed using any of a variety of components such as training data, model parameters, instruction sets, and software containers. These components can operate together to execute defined behaviors. Conventional approaches for versioning and identification of such components, or the AI system as a whole, often rely on naming schemes or decentralized tracking methods that cannot provide integrity or determinism across environments. When an AI agent is updated or deployed in multiple environments, reproducibility and provenance verification can become unreliable, since different infrastructure components can alter the underlying configuration or dependencies, or may lead to variations in the AI system operation. As such, it cannot be confirmed that a deployed AI agent corresponds to a reference version created by a manufacturer, resulting in challenges for validation or quality control. Without a verifiable linkage between component composition and running state, existing methods cannot establish a persistent identity for AI agents across manufacturing and deployment stages.

Systems and methods in accordance with the present disclosure can allow for more effective use of AI agents in any of a variety of applications, including but not limited to clinical workflows (e.g., reimbursements, acute care, pain scoring, education, patient support, etc.), conversational agents, customer support, robotic systems, vehicles, machine control, or various combinations thereof. This includes but is not limited to applications that have high performance criteria for AI system operation, such that non-verifiable AI or other unpredictable operations of the AI systems may not perform at a level to meet such criteria.

Systems and methods in accordance with the present disclosure can allow for a deterministic process for generating a verifiable identifier for an AI agent, where the verifiable identifier can be used to trigger deployment of the AI agent. This can include creating an agent fingerprint derived from the component structure of the AI agent. In some implementations, the system can generate a metadata file that includes canonicalized component identifiers representing data, model, instructions, and/or software components associated with a manufacturer definition. The metadata file can be encrypted, such as to be hashed using a one-way cryptographic function, such that the resulting agent fingerprint provides a stable and reproducible reference derived directly from the agent's manifest. In some implementations, a deployed agent fingerprint can be generated by extending the agent fingerprint with user-specific components including customer data or configuration details. The combination of the manufacturer's fingerprint and deployment-specific information can produce a deterministic blueprint that can be used by orchestration systems for deployment and by credentialing entities for validation of provenance and integrity. For example, the system can implement a technical mechanism to uniquely identify an AI agent based on the agent fingerprint, and can define the methods by which various systems that deploy or otherwise rely on the AI agent can technically communicate and interact. This identity can serve as the foundation for an automated governance pipeline, such as where an agent can be automatically evaluated against the standards of a specific agentic credential, receive a formal license to operate within a given jurisdiction, and be insured based on its verifiable qualifications, for example and without limitation. Systems and methods as described herein can be applied for any of a variety of AI systems, including any of various training and/or inference pipelines, machine learning models, neural networks, language models, multimodal models, agentic systems, retrieval systems, reinforcement models, or combinations thereof.

At least one aspect relates to a system. The system can identify an artificial intelligence (AI) agent comprising a plurality of components, the AI agent having an identifier for a manufacturer of the AI agent. The system can encrypt the plurality of components to obtain a plurality of component identifiers for the AI agent. The system can assemble the plurality of component identifiers and the identifier for the manufacturer into a structure of a metadata file. The system can encrypt the metadata file to generate a fingerprint of the AI agent. The system can provide the fingerprint and metadata file for validation of an instance of the AI agent by a user.

In some implementations, the plurality of components include at least a configuration of a model of the AI agent and a dataset that the model uses. In some implementations, the system can assemble the structure of the metadata file to include an encryption of the identifier for the manufacturer. In some implementations, the system can assemble the plurality of component identifiers and the identifier for the manufacturer into the structure of the metadata file according to a canonical arrangement for the plurality of component identifiers. In some implementations, the system can hash the metadata file to encrypt the metadata file. In some implementations, the structure of the metadata file comprises one or more name-value pairs, ordered lists, or comma-separated values that associate each component identifier of the plurality of component identifiers with a corresponding field. In some implementations, the system can provide the fingerprint as a blueprint of the manufacturer for a base deployment of the AI agent. In some implementations, the plurality of components include predefined prompts for the AI agent. In some implementations, the system can store the fingerprint and the metadata file as one or more tags on a representation of the AI agent in a registry. In some implementations, the system can generate the metadata file to include a manifest of one or more external dependencies for execution of the AI agent.

At least one other aspect relates to a system. The system can receive a metadata file and a fingerprint for an AI agent of a manufacturer to be deployed by a user. The system can identify a plurality of user-specific components to be used with the AI agent. The system can encrypt the plurality of user-specific components to obtain a plurality of component identifiers for the AI agent. The system can assemble the plurality of component identifiers and the metadata file into a structure of a deployment metadata file. The system can encrypt the deployment metadata file to generate a deployment fingerprint of the AI agent. The system can provide the deployment fingerprint and the deployment metadata file for validation of deployment of the AI agent with the user-specific components.

In some implementations, the AI agent comprises at least one of a neural network or a language model. In some implementations, the system can store at least one of the deployment fingerprint or the deployment metadata file in a version-controlled repository. In some implementations, the system can deploy the AI agent responsive to validation of the deployment. In some implementations, the system can generate the deployment metadata file to include a manifest of one or more external dependencies for deployment of the AI agent. In some implementations, the plurality of user-specific components include one or more data sources for use by the AI agent. In some implementations, the plurality of user-specific components include a user-specific configuration for deployment of the AI agent. In some implementations, the system can generate the deployment fingerprint to represent a blueprint for deployment of the AI agent using the user-specific components. In some implementations, the system can provide the deployment fingerprint as a configuration file for deployment of the AI agent.

At least one other aspect relates to a system. The system can receive a metadata file and a fingerprint for an AI agent of a manufacturer to be deployed by a user. The system can identify a plurality of user-specific components to be used with the AI agent. The system can encrypt the plurality of user-specific components to obtain a plurality of component identifiers for the AI agent. The system can assemble the plurality of component identifiers and the metadata file into a structure of a deployment metadata file. The system can encrypt the deployment metadata file to generate a deployment fingerprint of the AI agent. The system can provide the deployment fingerprint and the deployment metadata file for deployment of the AI agent with the user-specific components.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using any suitable apparatus, which may take the form of programmable computers running computer programs arranged to implement the aspect. As used in the specification and in the claims, the singular form of ‘a,’ ‘an,’ and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a block diagram of a system to generate an agent fingerprint, in accordance with one or more implementations;

FIG. 2 is a flow chart illustrating a method for generating an agent fingerprint, in accordance with one or more implementations;

FIG. 3 is a block diagram of an example of system to generate a deployed agent fingerprint, in accordance with one or more implementations; and

FIG. 4 is a flowchart illustrating a method for generating and validating a deployment fingerprint for an AI agent, in accordance with one or more implementations.

### DETAILED DESCRIPTION

Below are detailed descriptions of various concepts related to, and approaches, methods, apparatuses, and systems for implementing the various techniques described herein. The various concepts introduced above and discussed in greater detail below may be implemented in any of numerous ways, as the described concepts are not limited to any particular manner of implementation. Examples of specific implementations and applications are provided primarily for illustrative purposes.

The techniques described herein relate to systems and methods for generating deterministic identifiers for artificial intelligence (AI) agents, including deterministic hashing of components of the AI agents and/or of a pipeline for execution of the AI agents. AI agents can be executed or deployed using any one or more datasets, trained models, instruction sets, and executable software, which collectively can define the functional behavior of each agent. These agents can operate within distributed computing environments, enterprise networks, or cloud-based infrastructures. Verification of integrity for such agents can involve analyzing their component dependencies and configurations across manufacturing and deployment stages.

Conventional methods for identifying or tracking AI agents often rely on non-deterministic versioning schemes or manually assigned identifiers. These methods can lead to ambiguity when agents are updated, deployed across heterogeneous environments, or redeployed using modified configurations. Existing approaches may fail to establish a verifiable link between an identifier of an agent and the complete composition of the agent. As a result, validation systems cannot determine whether a deployed instance matches the original definition of that agent. Such limitations can create uncertainty in contexts that require compliance verification, credentialing, or reproducibility. For example, AI model cards can provide descriptive labels for AI agents, but are not verifiable (e.g., do not have cryptographic features to facilitate verification), and can lack a technical enforcement or verification mechanism for a deployed agent's full composition. Global identification schemas, such as MAC addresses, can uniquely identify hardware devices, but not complex, updatable software such as agents, and are not derived from the software composition of the devices. Resource identifiers such as FHIR can carry identifiers of data, but lack verification and adaptability functionality; for example, such identifiers operate as labels for data resources, not attestable fingerprints of executable agents-the identifier itself cannot provide a mechanism to prove the exact model version, RAG data, container image, and dependencies that constitute the running agent at a given moment, for example.

The techniques described herein can generate deterministic fingerprints for both a base AI agent (e.g., as provided by a manufacturer of the AI agent) and deployed variants of that agent. Each fingerprint can be derived from a structured metadata file that enumerates hashes of key components and their relationships. For example, an agent fingerprint can represent the canonical build for the agent, as produced by a manufacturer. A deployment fingerprint, such as a deployed agent fingerprint, can extend the identity by incorporating customer-specific data or configuration values. The combination of these fingerprints can provide a persistent and verifiable identity across manufacturing, deployment, and validation workflows. For example, a system of a deploying entity (whether a user, customer, and/or validating entity) can use at least one of the agent fingerprint or the deployed agent fingerprint as a verifiable source for components of the AI agent, such as to deterministically retrieve verified components of the agent and deploy the agent using the retrieved components. The agent fingerprint and/or deployed agent fingerprint can be a cryptographic derivative of the agent's actual components, e.g., of a real-time or as-deployed instance of the AI agent, which can allow for technical implementation of greater trust, attestation, and governance of the AI agent.

For example, the system can generate the agent fingerprint and/or deployed agent fingerprint to facilitate transparency and/or provenance, such as to provide an immutable bill of materials for any given AI agent. The system can provide a technical solution for trust and/or confidence in the AI agent, such as to enable rigorous and/or independent credentialing and licensing. The system can facilitate traceability and/or auditability, as each action executed by a deployed agent can be traced back to the deployed agent fingerprint, which can serve as the unique, verifiable identity for the deployed agent, (its DAF), which can create a clear audit trail. The system can facilitate accountability and/or liability; for example, by uniquely identifying an agent (as well as the provenance of its underlying components), liability for its actions can be more clearly assessed and risk adjusted, which can facilitate a technical implementation for a functional insurance market. The system can provide for greater reproducibility, as the fingerprinting mechanism can ensure that a certified agent's behavior can be precisely reproduced for testing, validation, and/or incident investigation. The system can allow for technical implementation of safety and/or recall functionality across deployments of AI agents, as the ability to uniquely identify every agent instance in the field can allow for targeted recalls or deactivations if a flaw or vulnerability is discovered.

A computing system implementing the techniques described herein can include one or more processors configured to hash and assemble component manifests into structured data objects, which can in turn be hashed to generate a machine-readable fingerprint for the AI agent. The system can create a metadata file containing component identifiers for data, model, instruction, and software elements. The metadata file can then be canonicalized and subjected to an encryption or hashing process, producing a deterministic fingerprint of the AI agent. For deployment, the system can receive additional user-defined components, generate new hashes for those components, and construct a deployment metadata file. Encryption of this deployment file can yield a deployment fingerprint that references the base agent fingerprint (e.g., to provide a provenance link to the AI agent as provided by the manufacturer), providing a verifiable linkage between the manufacturer's configuration and the deployed implementation. The resulting metadata structures can be stored in repositories (e.g., stored in artifact registries and tagged on container images) and used as configuration sources for automated orchestration or compliance evidence.

The techniques described herein can provide a reliable method for maintaining integrity, traceability, and/or reproducibility of AI agents throughout their lifecycle. By deriving fingerprints directly from canonicalized component data, the techniques eliminate inconsistencies introduced by environmental or human factors. Validation entities can verify that a running agent matches its declared configuration by recomputing the fingerprints from observed components. Deployment orchestrators can use the same metadata to instantiate consistent environments without manual intervention. Through these technical capabilities, the approaches described herein can establish a deterministic link between identity, configuration, and execution for AI agents.

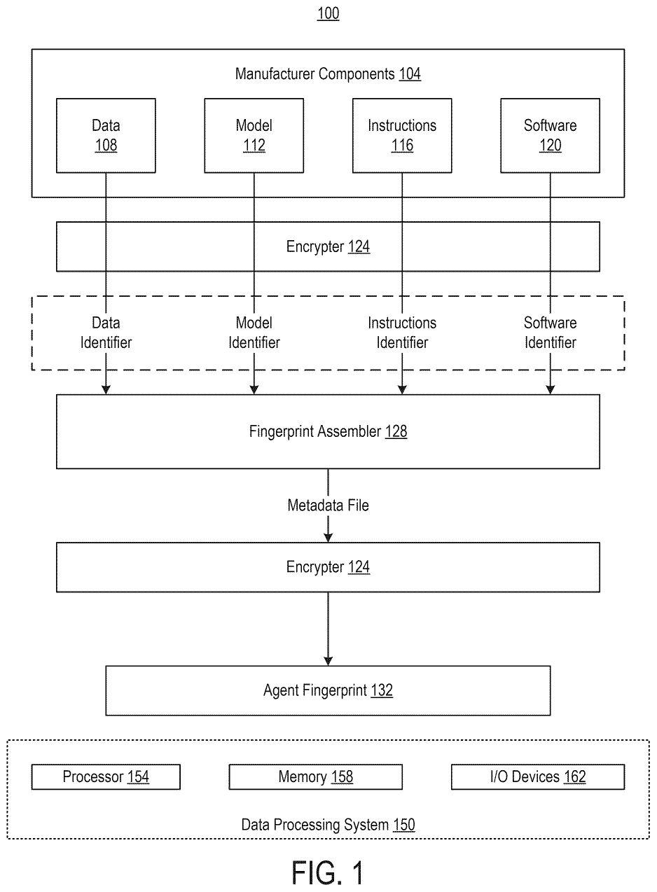

Referring now to FIG. 1, in brief overview, illustrated is a block diagram of a system 100, such as an agent fingerprint generation system 100. The system 100 can generate a deterministic identifier of an AI agent, derived from manufacturer components of the AI agent. For example, the system 100 can include manufacturer components 104, which can include data 108, model 112, instructions 116, and software 120. The system 100 can further include an encrypter 124 and a fingerprint assembler 128, and can output an agent fingerprint 132. The system 100 can include or be implemented using one or more data processing systems 150.

Referring to FIG. 1 in further detail, the system 100 can be or include a computing platform (e.g., data processing system 150) that processes component data of an AI agent to produce a deterministic fingerprint representing the AI agent. For example, the system 100 can be implemented as at least one of a cloud-based build pipeline or an on-premises system including one or more CPUs or GPUs. The system 100 can obtain component manifests, can generate hashes, and can assemble these into metadata files used to define an agent's identity. As an example, the system 100 can compute cryptographic digests for datasets, models, instruction files, and container images, which the system 100 can combine into a canonical metadata structure. The system 100 can perform deterministic hashing and metadata assembly procedures to compute a reproducible agent fingerprint based on canonical ordering rules. For example, the system 100 can use encryption, e.g., SHA-256 encryption, and can apply JSON canonicalization, to avoid environmental variance across builds. As depicted in FIG. 1, the data processing system 150 can include one or more processors 154, one or more memory (e.g., memory devices) 158, and/or one or more input/output (I/O) devices 162. The processor 154 can be a general purpose or specific purpose processor, an application specific integrated circuit (ASIC), one or more field programmable gate arrays (FPGAs), a group of processing components, or other suitable processing components. The processor 154 may be configured to execute computer code or instructions stored in memory (e.g., fuzzy logic, etc.) or received from other computer readable media (e.g., CDROM, network storage, a remote server, etc.) to perform one or more of the processes described herein. The memory 158 may include one or more data storage devices (e.g., memory units, memory devices, computer-readable storage media, etc.) configured to store data, computer code, executable instructions, or other forms of computer-readable information. The memory 158 may include random access memory (RAM), read-only memory (ROM), hard drive storage, temporary storage, non-volatile memory, flash memory, optical memory, or any other suitable memory for storing software objects and/or computer instructions. The processor 154 can be implemented as a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof. The memory 158 may include database components, object code components, script components, or any other type of information structure for supporting the various activities and information structures described in the present disclosure. The memory 158 can include various modules (e.g., circuits, engines) for completing processes described herein. The I/O devices 162 can include any one or more communications electronics (e.g., wired or wireless reception and/or transmission circuitry; communications busses; etc.), and can include any one or more user interface devices (e.g., displays, microphones, keyboards, mouse devices, touch input devices, etc.) to facilitate communication with the data processing system 150 and/or one or more components thereof. The data processing system 150 can be implemented in any of various computing platforms or architectures, including but not limited to any of various client-server architectures.

As illustrated in FIG. 1, the system 100 can include or obtain one or more components 104, such as manufacturer components 104 that a manufacturer of an AI agent provides to facilitate execution or deployment of the AI agent. The components 104 can be or include any one or more data structures, software, firmware, code, scripts, or pointers or identifiers thereof. The manufacturer can include an entity that develops, stores, or provides the components 104, e.g., via one or more repositories. For example, the manufacturer can be a manufacturer of record of the AI agent. The AI agent can include or be defined according to one or more of the components 104.

The AI agent can include one or more neural networks, language models, multimodal models, or combinations thereof, such as represented by model 112 of the components 104. The AI agent can be a self-contained computational entity that performs one or more tasks autonomously by processing data and executing algorithms that emulate human reasoning, decision-making, or problem-solving, for example and without limitation. The AI agent can include multiple interdependent components such as a trained model (e.g., trained model 112), data inputs (e.g., data 108), instructions (e.g., prompts and/or instructions 116), and/or executable software (e.g., software 120), which can determine or manage the operation or behavior of the AI agent. In some implementations, the AI agent can operate as a composite system integrating neural network architectures, reinforcement learning modules, and rule-based logic to generate context-dependent outputs. For example, a language-processing AI agent can receive textual inputs, can tokenize the inputs into interpretable elements, and can produce coherent responses derived from a parameterized transformer model. In some implementations, the AI agent can interact with external systems and data stores, can apply learned policies to dynamic environments, and can (continuously) update its internal state variables to optimize performance metrics. The AI agent can process numeric, categorical, linguistic, or multimodal data and can execute within a distributed infrastructure that includes cloud-based servers, local computing nodes, or edge devices. Each AI agent can maintain an internal configuration defining the parameters, data references, and operational contexts under which it performs assigned tasks, allowing deterministic instantiations for analysis, testing, and deployment across technical environments.

Referring further to FIG. 1, the components 104 can be elements (e.g., provided by the manufacturer) of a base definition of the AI agent. The base definition can represent, for example, a minimum or sufficient set of components 104 to allow for functionality of the AI agent (e.g., even if additional user data or other resources may be expected to be used to facilitate deployment of the AI agent according to one or more criteria of a user). For example, the components 104 may represent data files, trained models, instruction sets, and/or software containers, such as may be generated under controlled build conditions. The manufacturer components 104 can serve as the inputs from which individual component identifiers are computed for inclusion in the agent fingerprint, as described further herein.

The components 104 can include data 108. The data 108 can include any of various datasets, data manifests, or data assets that the AI agent (e.g., model 112) uses for training (including any of various unsupervised learning, supervised learning, fine-tuning, transfer learning, or in context-learning) and/or inference (including, for example and without limitation, for context data that the model 112 refers to in order to perform inference, or for retrieval, such as for retrieval-augmented generation (RAG)). For example, the data 108 may include structured medical datasets, language corpora, or tabular data used for specific domain adaptation. The data 108 can include any of various text, speech, audio, image, and/or video data. The data 108 can include structured or unstructured data. The data 108 can include training data pairs, such as training data elements that are each associated with respective labels.

The components 104 can include at least one model 112. The model 112 can include any one or more functions, algorithms, machine learning models, neural networks, reinforcement learning models, language models, large language models (LLMs), small language models (SLMs), multimodal language models, or combinations thereof. The model 112 can include data representing the structure of the model 112, such as any one or more configurations of the model 112, such as weights, biases, parameters, versions, base models, architectures, tuning parameters, arrangements or types of network layers, connections between layers, types of input data or output heads, or various combinations thereof. For example, the model 112 can represent a trained neural network or statistical model implementing an inferencing capability of the AI agent. For example, the model 112 may correspond to a large language model, a fine-tuned transformer, or other parametric architecture.

In some implementations, the components 104 include instructions 116. The instructions 116 can include at least one of instructions or prompts that the model 112 can process to perform corresponding actions. In some implementations, the model 112 uses at least a portion of instructions 116 as context. In some implementations, the model 112 can combine (e.g., append) one or more instructions 116 to prompts received from a user or other system to input to the model 112, e.g., to input to the AI agent The instructions 116 can represent operational control data or prompt templates directing behavior of the AI agent. For example, instructions 116 may be files including initialization prompts or system configurations guiding contextual responses.

In some implementations, the components 104 can include software 120. The software 120 can include or be coupled with or reference any one or more code, scripts or firmware, for example, that can execute the AI agent, such as to provide at least one of an application layer or an interface for or to the AI agent. For example, the software 120 can include executable code or a containerized image that packages runtime dependencies for the AI agent. For example, the software 120 can include container image digests that encapsulate environment variables, dependency libraries, and operating system layer hashes.

The agent fingerprint generation system 100 can include an encrypter 124. The encrypter 124 can include any one or more code, scripts, software, algorithms, functions, rules, or combinations thereof to perform operations such as applying an encryption, such as a cryptographic operation, on components 104. For example, the encrypter 124 can be a cryptographic engine, and can computes one or more hashes (e.g., hash values) of data inputted to the encrypter 124, such as to compute hashes of the respective components 104. In some implementations, the encrypter 124 includes a tokenizer or an embedding model. In some implementations, the encrypter 124 can implement a Federal Information Protection Standard (FIPS)-compliant algorithm to perform the encryption. The encrypter 124 can apply a cryptographic operation, such as SHA-256 or another deterministic hash function, to the components 104. For example, as described further herein, the encrypter 124 can receive a component 104 (e.g., any of the data 108, model 112, instructions 116, and/or software 120), and compute a hash (e.g., a cryptographic hash) of the component 104 to generate a corresponding identifier of the component (e.g., data identifier of the data 108; model identifier of the model 112; instructions identifier of the instructions 116, software identifier of the software 120). The encrypter 124 can generate the hashes to be encoded or encrypted representations of respective components 104, such as machine-readable representations.

In some implementations, the encrypter 124 hashes each of the components 104 to produce the identifiers as unique identifiers (which collectively can form the metadata record and/or metadata file). The encrypter 124 can ingest the components 104 for cryptographic derivation of the identifiers. As an example, the encrypter 124 can pass each file or manifest of the components 104 into an SHA-256 hashing process, to generate the respective identifier.

For example, the encrypter 124 can hash the data 108 to generate the data identifier. The data identifier can be a digest that represents a canonical form of the data 108. For example, the encrypter 124 may receive a manifest describing source repositories or data revisions, and can output a single cryptographic hash as the data identifier. The data identifier can be used by a deployment system to mount a correct, versioned dataset for the AI agent.

The encrypter 124 can hash the model 112 to generate the model identifier. The model identifier can convey the version and parameter configuration used in the build of the model 112. For example, the encrypter 124 can hash the model 112 to include the model architecture file and the configuration parameters of model 112 in a manifest represented by the model identifier, such as where a checksum of the manifest forms the model identifier. The model 112 may be input to the encrypter 124, which can compute the model identifier as a cryptographic hash of the model 112. The model identifier can be used by a deployment system to load the correct model files.

The encrypter 124 can hash the instructions 116 to generate the instructions identifier. The instructions identifier can be a unique identifier describing the instructions 116, such as where the instructions 116 include a version-controlled instruction set. For instance, the instructions identifier can be mapped to a version-controlled file (e.g., via Git commit hash) that is loaded by a deployment system at runtime.

The encrypter 124 can hash the software 120 to generate the software identifier. The software identifier can specify an exact executable environment for deployment of the AI agent. For example, the software identifier represent a specific and/or pullable container image digest (e.g., sha256: . . . ) that an orchestrator, such as Kubernetes, can used to deploy the exact software build for the AI agent. For example, the software identifier can be used as a direct and/or executable pointer.

Referring further to FIG. 1, the system 100 can include a fingerprint assembler 128. The fingerprint assembler 128 can include any one or more functions, algorithms, rules, policies, heuristics, models, or combinations thereof to perform operations such as to generate a data structure, such as a metadata file, based at least on the identifiers of the components 104 that the encrypter 124 generates. For example, the fingerprint assembler 128 can generate the metadata file according to a structure for the metadata file, such as an order of inclusion of data in fields of the metadata file. For example and without limitation, the structure can represent a set of name-value pairs, ordered lists, and/or comma-separated values. The fingerprint assembler 128 can generate the metadata file as a JSON file.

In some implementations, the fingerprint assembler 128 generates the metadata file to include an identifier of the manufacturer of the AI agent. For example, the fingerprint assembler 128 can use the encrypter 124 to hash a name or other identifier of the manufacturer to generate the identifier of the manufacturer. The fingerprint assembler 128 can generate the metadata file to include an identifier of the AI agent, such as a name or version of the AI agent; the identifier of the AI agent may be human-readable (or can be hashed to be a machine-readable identifier).

The fingerprint assembler 128 can generate the metadata file to include the identifiers of the components 104 from the encrypter 124. For example, the fingerprint assembler 128 can assemble the data identifier, model identifier, instructions identifier, and software identifier into the structure of the metadata file. By referencing the identifiers, the metadata file can allow for reproducible access to the components 104 (e.g., referencing the data identifier can allow for reproducible access to the datasets of the data 108 to use for operation of the AI agent).

The fingerprint assembler 128 can combine the identifiers into the structure of the metadata file, which can be used for agent validation and future deployments. The fingerprint assembler 128 can generate the metadata file to include data for one or more fields of the identifier of the manufacturer, the identifier of the AI agent, a timestamp indicating when the AI agent build was finalized, the identifiers of the components 104, and/or external dependency manifest data. The fingerprint assembler 128 can perform canonical ordering (e.g., according to a metadata schema) of the name-value pairs of such fields to prepare the structure for cryptographic hashing. As an example, the fingerprint assembler 128 can perform key ordering and array normalization steps to avoid nondeterministic serialization. In some implementations, the fingerprint assembler 128 generates the metadata file to include a version string for the metadata schema, which can further facilitate reliable use of the metadata file.

In some implementations, the fingerprint assembler 128 generates the metadata file to include (one or more identifiers of) one or more manifests of external dependencies for the AI agent. This can include, for example, an array declaring the types of external services the AI agent is architected to use, acting as a manifest of approved tool slots. The manifest of external dependencies can be used by a deployment system to pre-configure network access or service bindings, for example.

In some implementations, the fingerprint assembler 128 generates the metadata file to include (one or more identifiers of) an interface specification for the AI agent. For example, the fingerprint assembler 128 can include an identifier, such as a hash, of the interface specification (e.g., to a remote system, such as a model provider) that the dependency is to conform to, such as for automated client generation or interface testing.

The following is an illustrative example of a metadata file, including the identifiers and the schema for the metadata file:
{
“format_version”: “1.1”,
“manufacturer_id”: “87b1c428-2c67-428a-a195-21a4c4202c2e”,
“agent_model_name”: “MediBot-Nurse-v3.2-Intake”,
“timestamp”: “2025-08-27T10:00:00Z”,
“components”: {
“data_cid”:
“sha256:a1b2c3d4e5f678901234567890abcdef1234567890abcdef1234567890ab”,
“model_cid”:
“sha256: b2c3d4e5f6a1234567890abcdef1234567890abcdef1234567890abcde”,
“instructions_cid”:
“sha256:c3d4e5f6a1b234567890abcdef1234567890abcdef1234567890abcd”,
“software_cid”: “sha256:d4e5f6a1b2c34567890abcdef1234567890abcdef1234567890abcde”
},
“external_dependencies_manifest”: [
{
“dependency_id”: “EMBEDDINGS_PROVIDER”,
“description”: “Service for generating text embeddings for RAG.”,
   “interface_spec_cid”: “sha256:2b3c4d5e6f7a8901234567890abcdef1234567890abcdef1234567890”
},
{
“dependency_id”: “PATIENT_LOOKUP_API”,
“description”: “Tool for retrieving patient records from an EMR.”,
   “interface_spec_cid”: “sha256:3c4d5e6f7a2b901234567890abcdef1234567890abcdef123456789”
}
]
}

Referring further to FIG. 1, the encrypter 124 (which can be a same encrypter 124 that encrypts the components 104 into respective data, model, instructions, and/or software identifiers, or a different encrypter 124 or instance of an encrypter 124) can encrypt the metadata file to generate an agent fingerprint 132. For example, the encrypter 124 can hash the metadata file (e.g. and without limitation, using SHA256) to generate the agent fingerprint 132. The agent fingerprint 132 can be a cryptographic representation of the metadata file, such as to allow for a compact and/or verifiable representation of the AI agent and the components 104 used to deploy the AI agent.

The agent fingerprint 132 can represent the unique, deterministic identifier of the AI agent as produced by the manufacturer. For example, the agent fingerprint 132 can be the SHA-256 digest of the canonicalized AF metadata file describing all core components. The agent fingerprint 132 can be used as a verifiable reference or blueprint to validate any instance of the AI agent during deployment or credentialing. The system 100 can store the agent fingerprint 132 may be stored in a registry or deployment system for retrieval during later validation or licensing workflows. As an example, the system 100 can apply the agent fingerprint as a metadata tag, e.g., an immutable metadata tag, to a container image for the AI agent. This can allow the system 100 to facilitate verifiable use of the AI agent upon retrieval of the container image.

Referring further to FIG. 1, the system 100, e.g., using the encrypter 124, can generate component identifiers from manufacturer component inputs, can assemble the component identifiers (along with any of various other identifiers as noted above) into the structure of the metadata file, and can hash the assembled metadata file to form the agent fingerprint 132. For instance, the system 100 can first compute per-component identifiers, and can subsequently execute a final hash pass over the canonicalized JSON object produced by the fingerprint assembler 128. The encrypter 124 may perform these operations by serializing component manifests into a canonical format before computing digest outputs. As an example, canonicalization may include alphabetically ordering keys and removing extraneous whitespace prior to the hash computation.

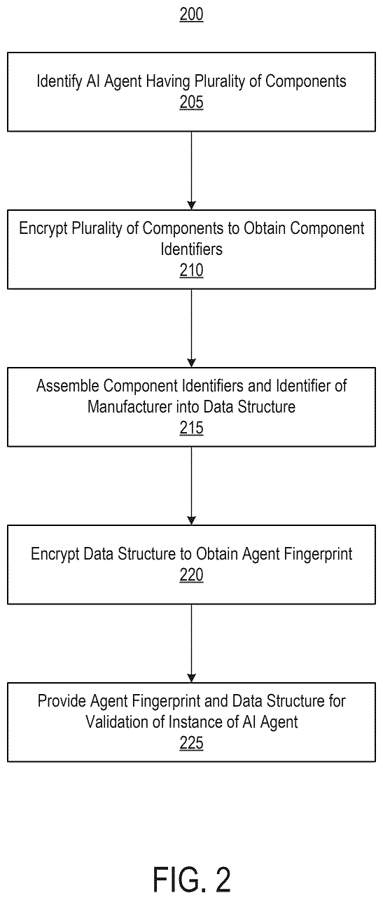

Referring now to FIG. 2, illustrated is a flow chart of a method 200 for generating an agent fingerprint for an AI agent. The method 200 can be executed, performed, or otherwise carried out by any of various systems described herein, including one or more components of the system 100. In brief overview of the method 200, the method 200 can include identifying an AI agent having a plurality of components 205, encrypting the plurality of components to obtain component identifiers 210, assembling the component identifiers and a manufacturer identifier into a data structure 215, encrypting the data structure to obtain an agent fingerprint 220, and providing the agent fingerprint and the data structure for validation of an instance of the AI agent 225. The method 200 or one or more operations of the method 200 can be triggered responsive to any of a variety of events, such as a request for validation or deployment of the AI agent, or in response to detection of a change in one or more components (e.g., components 104) of the AI agent.

At 205, the method 200 can include identifying an AI agent that includes or is associated with a plurality of components. For example, one or more manifests or repositories that define the constituent components of the AI agent can be accessed. Atomic elements for the AI agent such as data files, model parameters, instruction manifests, and/or executable software components can be identified, each representing a discrete element of the agent's operational configuration. In some implementations, version-controlled directories associated with a build environment of the AI agent can be processed to obtain resource descriptors that collectively define an operational scope for the agent. For example, the agent fingerprint generation system can enumerate structured references identifying model artifacts, data resources used for retrieval-augmented generation (RAG), container image references, and interface dependency manifests stored in respective repositories. The identification can occur at build initialization or when a manufacturer prepares a release candidate for fingerprint generation, such as during an automated continuous integration or continuous deployment pipeline that executes after source repositories containing model, data, and configuration definitions are checked out. The identification can occur responsive to a request to deploy or validate the AI agent.

At 210, the method 200 can include encrypting the plurality of components to obtain component identifiers of the plurality of components. Each component of the AI agent can be processed (e.g., encrypted, encoded) to generate a deterministic hash value, which can uniquely represent the data or content of the respective component content. In some implementations, a FIPS compliant cryptographic function such as Secure Hash Algorithm 256 (SHA-256) can be applied to each retrieved manifest for each component, which describes inputs including data, model, instructions, and/or software. For example, a base data manifest, a model configuration file, an instruction definition, and a container manifest can be read, serialized into a canonicalized form, and subjected to computation of corresponding cryptographic digests, thereby producing a set of component identifiers. In some implementations, canonicalization can involve ordering keys alphabetically, normalizing character encoding, and eliminating redundant whitespace to ensure that identical manifests always yield the same digest output. For example, a JSON metadata object representing a model configuration can be normalized prior to execution of the SHA-256 algorithm, which can produce a reproducible identifier value that accurately reflects the state of the model inputs within the manufacturing environment.

At 215, the method 200 can include assembling the component identifiers and an identifier of a manufacturer into a data structure, such as a data structure for a metadata file. For example, the identifiers of the components, an identifier of the manufacturer of the AI agent, and/or additional metadata such as timestamps or dependency manifests can be combined into a structured data object. A structured object can be generated as a metadata file containing name-value pairs that associate each identifier with a corresponding field defining the component type. In some implementations, identifiers of the data, model, instructions, and/or software for the AI agent can be merged into the metadata file. The assembly operation can be performed automatically after all component identifiers have been generated, thereby consolidating the component data into a complete and deterministic bill of materials. For example, in a continuous integration pipeline, the assembly can be triggered automatically at completion of the final component identifier hashing process to produce a finalized metadata object. The metadata file can be formatted in accordance with predefined canonicalization rules specifying serialization order and syntax consistency. In some implementations, alphabetical ordering of field names can be maintained, and consistent serialization can be applied across records to allow for reliable reproduction of identical cryptographic verification outputs across environments.

At 220, the method 200 can include encrypting the data structure (e.g., the metadata file) to obtain an agent fingerprint. For example, responsive to assembly of the metadata file, a cryptographic hash can be applied to the metadata file to generate the agent fingerprint, which can provide for a deterministic identifier of the AI agent and the components of the AI agent. In some implementations, the agent fingerprint can be expressed as AF=SHA-256(Canonicalize(AF_Metadata_File)), resulting in a reproducible digital signature derived from the canonicalized metadata content. The hashing operation can be performed after the assembly process is finalized, such as prior to the storage or distribution of the generated fingerprint. In some implementations, hashing can be executed during a final artifact packaging sequence within a continuous integration environment before release to a registry. The output of the hash computation can be verified through checksum comparison to detect any divergence between the computed value and expected reference data.

At 225, the method 200 can include providing the agent fingerprint and the data structure (e.g., the metadata file) for validation of an instance of the AI agent. For example, the agent fingerprint and the corresponding metadata file can be transmitted or otherwise made accessible to validation, credentialing, or licensing entities for reference in subsequent verification processes. In some implementations, the generated data can be uploaded to a credential-granting organization or deposited within an attestation registry that permits access by authorized verification systems. For example, completion of fingerprint generation and archival operations can precede the transmission phase, allowing the information to be used as a reference record for deployment validation or credential assessment. In some implementations, distribution of the fingerprint and metadata can occur during the final build stage to align the release of the agent with the initiation of credential verification procedures. The fingerprint and metadata can be provided through one or more secure interfaces that facilitate retrieval and comparison of encrypted component identifiers. For example, a verification authority can load a corresponding deployed container image, regenerate an associated fingerprint, and confirm alignment with the manufacturer-declared fingerprint to validate authenticity of the agent instance.

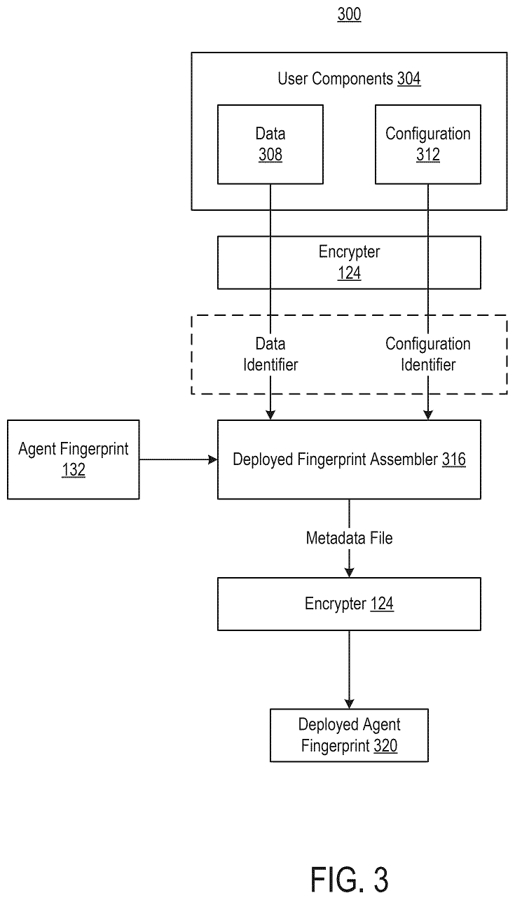

Referring now to FIG. 3, illustrated is a block diagram of a system 300, such as a system for generating a deployed agent fingerprint for a deployed instance of an AI agent. The AI agent can correspond to the AI agent described with reference to FIG. 1, where the deployed instance can be further updated (e.g., customized, modified, trained, fine-tuned, etc.) for a target application, such as for a customer and/or user of the AI agent (e.g., in contrast to the base definition of the AI agent that may be represented by the components 104 and/or agent fingerprint 132). The system 300 can be implemented to generate the deployed agent fingerprint based on customer-specific components and a previously established agent fingerprint. In some implementations, the system 300 obtains the agent fingerprint 132 that the system 100 generates, for example. In brief overview, the system 300 can include one or more user components 304, the encrypter 124, and a deployed fingerprint assembler 316, and can output a deployed agent fingerprint 320. The user components 304 can include data 308 and configuration 312.

As depicted in FIG. 3, the system 300 can include or obtain one or more components 304, which can be user components 304. For example, the components 304 can represent deployment-specific assets supplied by a user, customer, or end organization for use with the AI agent. For example, the user components 304 can include customer-provided data sources, configuration files, and local environment manifests utilized during deployment. The AI agent can use the components 304 to deploy a user-specific (e.g., customer-specific) instance of the AI agent.

The components 304 can include data 308. The data 308 can include one or more datasets or resource manifests for the AI agent. In some implementations, the data 308 includes domain-specific information and/or data for RAG operations that the AI agent is to perform. For example, the data 308 can include medical records datasets, financial policy tables, or other proprietary information local to the deploying organization. The data 308 can include data from or identifiers of any of various data sources.

The user components 304 can include at least one configuration 312. The configuration 312 can include one or more files or manifests that define parameters and/or settings for the deployment of the AI agent. For example, the configuration 312 can specify parameters such as environment variables, model bindings, or resource allocation limits unique to the target environment (e.g., software and/or hardware environment) in which the AI agent is to be deployed.

Referring further to FIG. 3, the system 300 can include the encrypter 124. The encrypter 124 can be configured for the system 300 in a manner analogous to the encrypter 124 of the system 100 (though may be implemented using one or more separate encrypters or encryption functions than used by the system 100). The encrypter 124 can encrypt the components 304 to generate identifiers of the components 304, such as to compute respective hashes of the data 308 and/or configuration 312 to generate a data identifier and/or a configuration identifier. As an example, each asset within the components 304 can be hashed to form identifiers which, as described further herein, the system 300 can append to a metadata file referencing the agent fingerprint.

For example, the encrypter 124 can hash the data 308 form a data identifier, which can represent the dataset to be used in deployment of the AI agent. As an example, the encrypter 124 may compute a SHA-256 digest for a customer data manifest represented by the data 308, which can yield a unique value linked to the corresponding data 308. The data identifier can be a hash of the manifest listing all customer-specific RAG documents or other local data sources. The data identifier can be used by as deployment system to mount the data 308. In some implementations, the encrypter 124 serializes the data 308 before hashing, which can maintain deterministic reproducibility of dataset references. For instance, canonicalization may involve key sorting or format normalization prior to digest computation to ensure consistency across environments.

The encrypter 124 can hash the configuration 312 to generate a configuration identifier, which can represent the customer- (or user) specific settings for deployment of the AI agent. For example, the configuration identifier can be consumed by the deployment system to apply environment variables, feature flags, or resource limits to the deployment of the AI agent. In some implementations, the configuration 312 may be encoded in a canonicalized JSON or YAML structure before processing by the encrypter 124. For instance, the structure can be flattened and normalized before hashing to prevent variations due to whitespace or system differences.

Referring further to FIG. 3, the system 300 can include a deployed fingerprint assembler 316. The deployed fingerprint assembler 316 can be analogous to or include components and/or functionality of the fingerprint assembler 128. The deployed fingerprint assembler 316 can generate a deployment metadata file that includes the agent fingerprint 132 and the hashed identifiers for user-specific components. The deployed fingerprint assembler 316 can perform data processing operations that merge these values into a canonical structure ready for encryption. In some implementations, the deployed fingerprint assembler 316 can construct a JSON object that defines fields such as for the agent fingerprint, the identifier of the data 308, and/or the identifier of the configuration 312. For example, the deployed fingerprint assembler 316 can retrieve the agent fingerprint 132, combine it with the computed identifiers from the encrypter 124, and assemble the combined fields into a structured object. In some implementations, the deployed fingerprint assembler 316 can apply canonicalization routines to establish consistent field ordering across computing environments. For example, the deployed fingerprint assembler 316 can alphabetically sort field names, normalize data types, and validate compliance with a predefined schema before passing the metadata to the encrypter 124 for hashing. The resulting canonical metadata file can be serialized using a deterministic encoding format so that identical logical content produces the same binary representation during subsequent encryption operations.

In some implementations, the deployed fingerprint assembler 316 generates the metadata file to include (one or more identifiers of) one or more deployment dependencies. For example, the identifier(s) can include an array of objects that can indicate an implementation of each corresponding dependency identifier declared in the agent fingerprint. The identifiers of the deployment dependencies can be used by the deployment orchestrator to configure network policies, firewall rules, or service mesh routes, for example and without limitation, such as to ensure that the AI agent can only communicate with its declared dependencies.

The following is an illustrative example of the metadata file generated by the deployed fingerprint assembler 316, including the identifiers and the schema for the metadata file:
{
“format_version”: “1.”,
“manufacturer_id”: “87b1c428-2c67-428a-a195-21a4c4202c2e”,
“agent_model_name”: “MediBot-Nurse-v3.2-Intake”,
“timestamp”: “2025-08-27T10:00:00Z”,
“components”: {
   “data_cid”: “sha256:a1b2c3d4e5f678901234567890abcdef1234567890abcdef1234567890ab”,
   “model_cid”: “sha256:b2c3d4e5f6a1234567890abcdef1234567890abcdef1234567890abcde”,
   “instructions_cid”: “sha256:c3d4e5f6a1b234567890abcdef1234567890abcdef1234567890abcd”,
   “software_cid”: “sha256:d4e5f6a1b2c34567890abcdef1234567890abcdef1234567890abcde”
   },
   “external_dependencies_manifest”: [
   {
   “dependency_id”: “EMBEDDINGS_PROVIDER”,
   “description”: “Service for generating text embeddings for RAG.”,
      “interface_spec_cid”: “sha256:2b3c4d5e6f7a8901234567890abcdef1234567890abcdef1234567890”
   },
   {
   “dependency_id”: “PATIENT_LOOKUP_API”,
   “description”: “Tool for retrieving patient records from an EMR.”,
      “interface_spec_cid”: “sha256:3c4d5e6f7a2b901234567890abcdef1234567890abcdef123456789”
   }
   ]
   }

Referring further to FIG. 3, the system 300 can use the encrypter 124 to generate a deployed agent fingerprint 320 based on the metadata file (e.g., the metadata file generated by the deployed fingerprint assembler 316). The deployed agent fingerprint 320 can represent a deterministic cryptographic identifier associated with a specific deployment instance of an AI agent that incorporates customer-specific components. In some implementations, the deployed agent fingerprint 320 can be generated by hashing, e.g., applying a cryptographic hash function such as SHA-256, to a canonicalized deployment metadata file representing the deployment-related parameters. For example, the system 300 can compute the fingerprint as SHA-256(Canonicalize(DAF_Metadata_File)), thereby producing a reproducible identifier that establishes a verifiable association between the manufacturer definition and the customer deployment instance. In some implementations, the deployed agent fingerprint 320 can be transmitted to one or more validation systems, which can confirm component alignment and provenance across distinct customer environments. For example, a credential-granting authority can recompute a fingerprint directly from a deployed image and compare its value to the declared deployed agent fingerprint 320 to confirm that the deployment precisely matches the expected configuration. The deployed agent fingerprint 320 can be retained in a version-controlled repository or deployment register to facilitate subsequent verification or attestation of a specific release version. For example, the fingerprint can be stored as an immutable metadata tag linked to a corresponding container image of the AI agent in an artifact registry, establishing a persistent record of the deployed artifact for future reference.

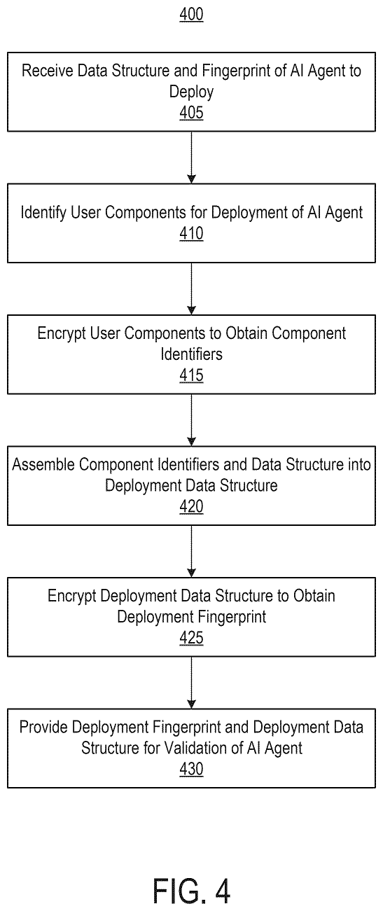

Referring now to FIG. 4, illustrated is a flowchart of a method 400 for generating and validating a deployment fingerprint for an AI agent. The method 400 can be executed, performed, or otherwise carried out by any of the computing systems described herein. In brief overview of the method 400, the method 400 can include receiving a data structure (e.g., metadata file) and fingerprint of AI agent to deploy 405, identifying user components for deployment of AI agent 410, encrypting user components to obtain component identifiers 415, assembling component identifiers and data structure into deployment data structure 420, encrypting deployment data structure to obtain a deployment fingerprint 425, and providing the deployment fingerprint and deployment data structure for validation of AI agent 430.

At 405, a data structure and a fingerprint of an AI agent to be deployed can be received. The data structure can include a metadata file, such as a metadata file defining a base configuration of the AI agent. In some implementations, the data structure and fingerprint can be obtained from a repository maintained by a manufacturer of record prior to deployment initialization. For example, retrieval can occur automatically through a continuous deployment pipeline, such as when a change to a deployment configuration is detected in a version control repository. The data structure and fingerprint can be accessed through secured interfaces, such as application programming interfaces or encrypted manifests, and verified for integrity through signed artifact exchange.

At 410, user-specific components required for deployment of the AI agent can be identified. The components can include customer data inputs and configuration files that define the deployment environment. In some implementations, the identification can occur after validation of the manufacturer's metadata file to confirm compatibility between manufacturer and customer-specific component definitions. For example, reference manifests can be inspected to locate resource files corresponding to domain-specific datasets and configuration templates. Identifiers, version descriptors, or asset paths defining each customer element can be determined to prepare for subsequent encryption of the user components.

At 415, encryption of the user-specific components can be performed to obtain component identifiers. Each deployment-specific manifest can be processed as an input to a cryptographic hashing operation. In some implementations, the user-specific components can be serialized and canonicalized before encryption to maintain deterministic output across environments. For example, manifest files can be normalized by ordering keys alphabetically and standardizing character encoding before computation of the hash values. The resulting identifiers can uniquely represent the data and configuration information used for deployment of the artificial intelligence agent.

At 420, the component identifiers and the received manufacturer data structure can be assembled into a deployment data structure. The deployment data structure can include combined fields, such as a base agent fingerprint, a customer data identifier, and a customer configuration identifier. In some implementations, the assembly process can occur automatically after all customer-specific hashing operations are completed. For example, a build pipeline may generate a JSON file once the component identifier values have been determined. Canonicalization steps can then be applied to ensure a deterministic representation, such as by sorting keys or aligning nested arrays according to schema specifications prior to serialization.

At 425, the deployment data structure can be encrypted to obtain a deployment fingerprint. The deployment fingerprint can result from a cryptographic hash computation that converts the canonicalized deployment data into a deterministic identifier. In some implementations, the fingerprint can be expressed as DAF=SHA-256(Canonicalize(DAF_Metadata_File)). For example, cryptographic hashing can be performed during a final build stage of a deployment pipeline to produce an immutable release artifact. Whitespace removal and/or numeric normalization can be applied to the data structure before execution of the hashing procedure to maintain reproducibility of the output fingerprint across processing environments.

At 430, the deployment fingerprint and the associated deployment data structure can be provided for validation of the AI agent. The data can be made available to a credentialing or licensing entity for verification. In some implementations, the fingerprint and deployment data structure can be submitted as final artifacts within a deployment pipeline prior to instantiation of the agent within a compute cluster. For example, the data can be transmitted as signed objects through secure interfaces such as application programming interfaces to enable integrity comparison against a reference deployment fingerprint computed independently. Validation can include comparison of fingerprints to confirm alignment between the declared configuration and the deployed instance.

Systems and methods as described herein can be implemented by any of various neural networks and/or machine learning models. These can include, for example and without limitation, one or more neural networks (or layers, nodes, weights, and/or biases thereof), convolutional neural networks, recurrent neural networks, attention networks, transformer networks, encoders, decoders, sequence to sequence models, generative models, pretrained models, diffusion models, multimodal models, generative adversarial networks, or various combinations thereof, which may be configured (e.g., trained, fine-tuned, having transfer learning performed, updated or operated by in-context learning, examples, or prompting, etc.) through operations such as supervised learning, self-supervised learning, or unsupervised learning. Systems and methods as described herein can be implemented in any of various artificial intelligence architectures or processing pipelines, including, for example, agentic pipelines, retrieval-based pipelines (e.g., retrieval-augmented generation), or various combinations thereof.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the implementations disclosed herein can be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor can be a microprocessor, or, any conventional processor, controller, microcontroller, soc (system on chip), som (system on module) or state machine. A processor also can be implemented as a combination of computing devices, such as a combination of a DSP and a microprocessor, a plurality of microprocessors, one or more microprocessors in conjunction with a DSP core, or any other such configuration. In some implementations, particular processes and methods can be performed by circuitry that is specific to a given function. The memory (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present disclosure. The memory can be or include volatile memory or non-volatile memory, and can include database components, object code components, script components, or any other type of information structure for supporting the various activities and information structures described in the present disclosure. According to an exemplary implementation, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The implementations of the present disclosure can be implemented using existing computer processors, or by a special purpose computer processor for an appropriate system, incorporated for this or another purpose, or by a hardwired system. Implementations within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or implementation, and references to “an implementation,” “some implementations,” “one implementation” or the like are not necessarily mutually exclusive and are intended to indicate that a particular feature, structure, or characteristic described in connection with the implementation can be included in at least one implementation or implementation. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein can be embodied in other specific forms without departing from the characteristics thereof. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining can be stationary (e.g., permanent or fixed) or moveable (e.g., removable or releasable). Such joining can be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling can be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements can differ according to other exemplary implementations, and that such variations are intended to be encompassed by the present disclosure.

What is claimed is:

1. A system of artificial intelligence (AI) agent fingerprinting, comprising:
one or more processors to:
identify an AI agent comprising a plurality of components, the AI agent having an identifier for a manufacturer of the AI agent;
encrypt the plurality of components to obtain a plurality of component identifiers for the AI agent;
assemble the plurality of component identifiers and the identifier for the manufacturer into a structure of a metadata file;
encrypt the metadata file to generate a fingerprint of the AI agent; and
provide the fingerprint and the metadata file for validation of an instance of the AI agent by a user.

2. The system of claim 1, wherein the plurality of components include at least a configuration of a model of the AI agent and a dataset that the model uses.

3. The system of claim 1, wherein the one or more processors are to assemble the structure of the metadata file to include an encryption of the identifier for the manufacturer.

4. The system of claim 1, wherein the one or more processors are to assemble the plurality of component identifiers and the identifier for the manufacturer into the structure of the metadata file according to a canonical arrangement for the plurality of component identifiers.

5. The system of claim 1, wherein the one or more processors are to hash the metadata file to encrypt the metadata file.

6. The system of claim 1, wherein the structure of the metadata file comprises one or more name-value pairs, ordered lists, or comma-separated values that associate each component identifier of the plurality of component identifiers with a corresponding field.

7. The system of claim 1, wherein the one or more processors are to provide the fingerprint as a blueprint of the manufacturer for a base deployment of the AI agent.

8. The system of claim 1, wherein the plurality of components include predefined prompts for the AI agent.

9. The system of claim 1, wherein the one or more processors are to store the fingerprint and the metadata file as one or more tags on an representation of the AI agent in a registry.

10. The system of claim 1, wherein the one or more processors are to generate the metadata file to include a manifest of one or more external dependencies for execution of the AI agent.

11. A system comprising:
one or more processors to:
receive a metadata file and a fingerprint for an AI agent of a manufacturer to be deployed by a user;
identify a plurality of user-specific components to be used with the AI agent;
encrypt the plurality of user-specific components to obtain a plurality of component identifiers for the AI agent;
assemble the plurality of component identifiers and the metadata file into a structure of a deployment metadata file;
encrypt the deployment metadata file to generate a deployment fingerprint of the AI agent; and
provide the deployment fingerprint and the deployment metadata file for validation of deployment of the AI agent with the plurality of user-specific components.

12. The system of claim 11, wherein the one or more processors are to further provide the fingerprint of the AI agent for validation of the AI agent with the plurality of user-specific components.

13. The system of claim 11, wherein the one or more processors are to store at least one of the deployment fingerprint or the deployment metadata file in a version-controlled repository.

14. The system of claim 11, wherein the one or more processors are to deploy the AI agent responsive to validation of the deployment.

15. The system of claim 11, wherein the one or more processors are to generate the deployment metadata file to include a manifest of one or more external dependencies for deployment of the AI agent.

16. The system of claim 11, wherein the plurality of user-specific components include one or more data sources for use by the AI agent.

17. The system of claim 11, wherein the plurality of user-specific components include a user-specific configuration for deployment of the AI agent.

18. The system of claim 11, wherein the one or more processors are to generate the deployment fingerprint to represent a blueprint for deployment of the AI agent using the plurality of user-specific components.

19. The system of claim 11, wherein the one or more processors are to provide the deployment fingerprint as a configuration file for deployment of the AI agent.

20. A system comprising:
one or more processors to:
receive a metadata file and a fingerprint for an AI agent of a manufacturer to be deployed by a user;
identify a plurality of user-specific components to be used with the AI agent;
encrypt the plurality of user-specific components to obtain a plurality of component identifiers for the AI agent;
assemble the plurality of component identifiers and the metadata file into a structure of a deployment metadata file;
encrypt the deployment metadata file to generate a deployment fingerprint of the AI agent; and
provide the deployment fingerprint and the deployment metadata file for deployment of the AI agent with the plurality of user-specific components.
